(19) United States
(12) Patent Application Publication
(10) Pub. No.: US 2024/0157044 A1
(43) Pub. Date: May 16, 2024

(54) PNEUMATIC INJECTOR FOR MEDICINE DELIVERY

(71) Applicant: Bigfoot Biomedical, Inc., Milpitas, CA (US)

(72) Inventor: Peter Kimball, Milpitas, CA (US)

(21) Appl. No.: 18/509,123

(22) Filed: Nov. 14, 2023

Related U.S. Application Data
(60) Provisional application No. 63/383,630, filed on Nov. 14, 2022.

(51) Int. Cl. A61M 5/145; A61M 5/172

(52) U.S. Cl. CPC A61M 5/14526; A61M 5/172; A61M 2005/1403

(57) ABSTRACT

One or more examples may include a system. Such a system may include a cartridge, a pneumatic drive system, a position detector, and a controller. The cartridge may include a piston configured to move within a holding chamber of the cartridge and urge medicine toward an interface between the holding chamber and a region external to the cartridge. The pneumatic drive system may be configured to actuate movement of the piston within the holding chamber. The position detector may be configured to generate information about a position of the piston within the holding chamber. The controller may be configured to vent working fluid from the pneumatic drive system at least partially responsive to information about the position of the piston within the holding chamber generated by the position detector.

### CROSS-REFERENCE TO RELATED APPLICATION

This application claims the benefit under 35 U.S.C. § 119(e) of U.S. Provisional Patent Application Ser. No. 63/383,630, filed Nov. 14, 2022, the disclosure of which is hereby incorporated herein in its entirety by this reference.

### FIELD

One or more examples relate, generally, to a pneumatic drive system for an automatic injector—a “new” pneumatic injector.

### BACKGROUND

Many chronic disease patients are prescribed medications that need to be self-administered using injection pens or similar drug delivery devices. For example, persons diagnosed with Type I or II diabetes need to regularly check their blood glucose levels and self-administer an appropriate dose of insulin using an injection pen.

### BRIEF DESCRIPTION OF THE DRAWINGS

To easily identify the discussion of any particular element or act, the most significant digit or digits in a reference number refer to the figure number in which that element is first introduced.

FIG. 1 is a schematic diagram to deliver medicine, in accordance with one or more examples.

FIG. 2 is a flow chart of a process for controlling venting at pneumatic drive system of a medicine injector, in accordance with one or more examples.

FIG. 3 is a flow chart of a process for inhibiting operation of pneumatic drive system of a medicine injector, in accordance with one or more examples.

FIG. 4 is a flow chart of a process for inhibiting operation of pneumatic drive system of a medicine injector, in accordance with one or more examples.

FIG. 5 is a flow chart of a process for inhibiting operation of pneumatic drive system of a medicine injector, in accordance with one or more examples.

### DETAILED DESCRIPTION

In the following detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

The illustrations presented herein are not meant to be actual views of any particular method, system, device, or structure, but are merely idealized representations that are employed to describe the embodiments of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The following description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawing could be arranged and designed in a wide variety of different configurations. Thus, the following description of various embodiments is not intended to limit the scope of the present disclosure, but is merely representative of various embodiments. While the various aspects of the embodiments may be presented in drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative logical blocks, modules, and circuits described in connection with the embodiments disclosed herein may be implemented or performed with a general purpose processor, a special purpose processor, a Digital Signal Processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to embodiments of the present disclosure.

The embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, without limitation. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

Any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may comprise one or more elements.

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

As used herein, any relational term, such as “over,” “under,” “on,” “underlying,” “upper,” “lower,” without limitation, is used for clarity and convenience in understanding the disclosure and accompanying drawings and does not connote or depend on any specific preference, orientation, or order, except where the context clearly indicates otherwise.

In this description the term “coupled” and derivatives thereof may be used to indicate that two elements co-operate or interact with each other. When an element is described as being “coupled” to another element, then the elements may be in direct physical or electrical contact or there may be intervening elements or layers present. In contrast, when an element is described as being “directly coupled” to another element, then there are no intervening elements or layers present. The term “connected” may be used in this description interchangeably with the term “coupled,” and has the same meaning unless expressly indicated otherwise or the context would indicate otherwise to a person having ordinary skill in the art.

As used herein, the terms “assert,” “de-assert” and derivatives thereof used in reference to a pin, means, respectively, to assert or de-assert a signal associated with the pin (e.g., a signal specifically assigned to the pin or a signal to which the pin is specifically assigned, without limitation).

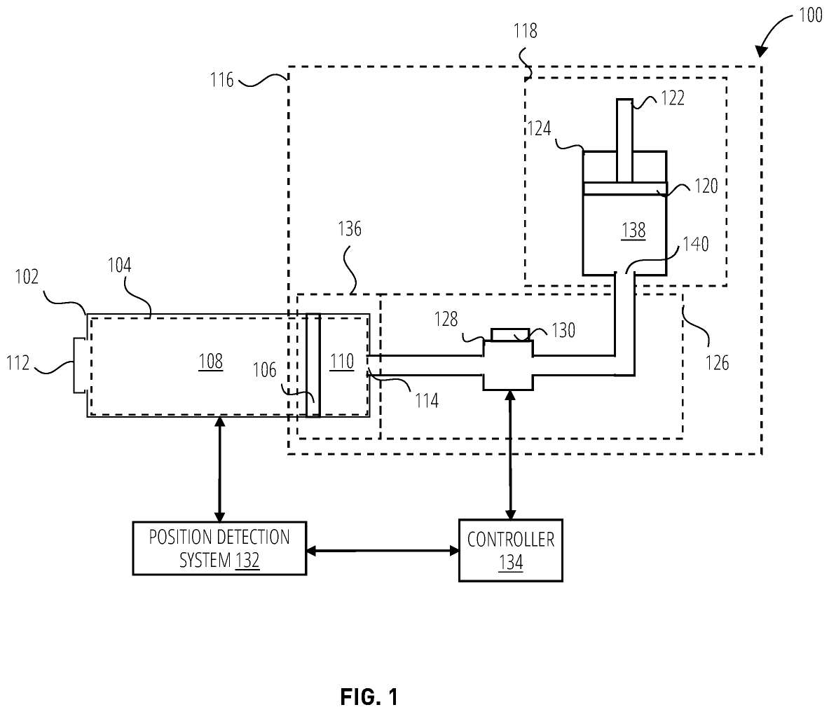

FIG. 1 is a schematic diagram depicting a system 100 to deliver medicine, in accordance with one or more examples.

System 100 includes a cartridge 102, a pneumatic drive system 116, position detection system 132, and a controller 134.

Cartridge 102 is configured, generally, to hold and, when operated, dispense medicine.

Cartridge 102 includes piston 106, first port 112, and second port 114. Piston 106 is movably located within interior chamber 104 of cartridge 102. A first space 108 of interior chamber 104 is defined in front of piston 106 and a second space 110 of interior chamber 104 is defined behind piston 106. Respective volumes of first space 108 and second space 110 change in response to movement of piston 106. In one or more examples, first space 108 may be utilized to hold medicine and may be referred to herein as a “holding chamber.” In one or more examples, second space 110 may be utilized to hold working fluid of pneumatic drive system 116 as discussed below. Piston 106 is configured to move within the interior chamber of cartridge 102 and urge medicine in first space 108 of interior chamber 104 toward and/or through first port 112.

First port 112 is configured as an interface between interior chamber 104, and more specifically, first space 108, and a region external to cartridge 102. First port 112 may, during operation of cartridge 102, put a sharp (e.g., hypodermic needle, without limitation) in fluid communication with first space 108 of interior chamber 104.

Second port 114 is configured as an interface between interior chamber 104, and more specifically second space 110, and a region external to cartridge 102.

Piston 106 is dimensionally sized and shaped to match a cross-section of the interior chamber of cartridge 102 and to permit movement of piston 106 in response to an urging force within the interior chamber. Piston 106 is arranged within the interior chamber of cartridge 102 to extend across the interior chamber of cartridge 102 and form an airtight seal with an interior wall of cartridge 102. In one or more examples, piston 106 may be substantially disc-shaped.

Pneumatic drive system 116 is configured to actuate movement of piston 106 within interior chamber 104 of cartridge 102, and more specifically, to actuate a degree of movement to dispense medicine from cartridge 102, as discussed below.

FIG. 1 depicts second space 110 of first space 108 and piston 106 as being of cartridge 102 and as being of pneumatic drive system 116. A person having ordinary skill in the art will appreciate that piston 106 and second space 110 may be considered parts of cartridge 102, pneumatic drive system 116, or common parts of cartridge 102 and pneumatic drive system 116 without exceeding the scope of this disclosure.

Pneumatic drive system 116 includes compressor 118, drive mechanism 136, and fluid transfer system 126. Compressor 118 is configured to generate working fluid. Compressor 118 may be any suitable device or system for generating working fluid. Pneumatic drive system 116 may utilize any suitable working fluid. Non-limiting examples of working fluid include compressed air or other suitable compressed gas or expanding gas. Drive mechanism 136 is configured to apply a force to piston 106 of cartridge 102. Fluid transfer system 126 is configured to provide working fluid to and from the drive mechanism, and more specifically, provide working fluid from tank 124 of compressor 118 to drive mechanism 136.

In one or more examples, compressor 118 may include a mechanism to move and reset a position of mechanical button 122 and piston 120 at or near the top of tank 124 (as depicted by FIG. 1) and thereby draw pre-working fluid (e.g., air, without limitation) into region 138 of tank 124 (intake port not depicted).

In one or more examples, compressor 118 may be or include a click-action device, and mechanical button 122 and piston 120 may be part of the same. During a first click action the mechanical button 122 and piston 120 extend (e.g., under force from manual actuation of the mechanical button 122, without limitation) toward port 140 and stay in a locked, but impermanent, position near port 140. During a second click-action, the mechanical button 122 and piston 120 retract toward the opposite side of tank 124 as the side including port 140.

Fluid transfer system 126 includes vent 130 and valve 128. Vent 130 is configured to permit escape of working fluid from fluid transfer system 126 or fluid transfer system 126 and second space 110 as discussed herein. Valve 128 is configured to regulate flow of working fluid to vent 130. Valve 128 is configured to exhibit at least two states: an open state and a closed state. the open state is associated with permitting flow of working fluid from the fluid transfer system 126 to vent 130. The closed state is associated with inhibiting or blocking flow of working fluid from the fluid transfer system 126 to vent 130.

Fluid transfer system 126 may include a drive mechanism configured to, at least partially responsive to a command to vent working fluid, change a state of valve 128 from the first state to the second state, and change a state of valve 128 from the second state to the first state.

Position detection system 132 is configured to generate information about a position of piston 106 within the interior chamber 104 of cartridge 102. In one or more examples, position detection system 132 may be or include an optical sensor, capacitive sensor, or inductive sensor, without limitation.

Controller 134 is configured to vent working fluid from pneumatic drive system 116 at least partially responsive to information about the position of piston 106 within interior chamber 104 of cartridge 102 generated by position detection system 132.

In one or more examples, system 100 may include an inhibitor to selectively inhibit operation of the pneumatic drive system. When enabled, such an inhibitor may inhibit operation of pneumatic drive system 116. Non-limiting examples of inhibiting operation of pneumatic drive system 116 include inhibiting movement of mechanical button 122, piston 120, or both, closing, and holding closed, one or more valves in the passageways of fluid transfer system 126 utilized to convey working fluid to drive mechanism 136; or opening, and holding opening, valve 128 of fluid transfer system 126.

In one or more examples, controller 134 may be configured to alternately enable and disable such an inhibitor at least partially in response to detecting improper operation of pneumatic drive system 116. Non-limiting examples of improper operation may include improper venting of pneumatic drive system 116, improper positioning of piston 106 within interior chamber 104 of cartridge 102, or both.

In one or more examples, controller 134 may detect improper operation of pneumatic drive system 116 at least partially responsive to information about a positioning of piston 106 within interior chamber 104 generated by the position detection system 132.

As a non-limiting example, controller 134 may detect improper positioning of piston 106 within interior chamber 104 of cartridge 102 at least partially responsive to information about a positioning of piston 106 generated by position detection system 132. The information about positioning of piston 106 may include an indicated position that is different than a target position associated with a predetermined amount of medicine (e.g., a dose of medicine, without limitation). Such an indicated position may be ahead of, or behind, the target position, and in either case, controller 134 cannot confirm that a desired amount of medicine was delivered by system 100 because the indicated position of piston 106 is different than the target position of piston 106.

In one or more examples, controller 134 may detect improper venting of pneumatic drive system 116 at least partially responsive to information about a positioning of piston 106 within interior chamber 104 generated by the position detection system 132. As a non-limiting example, the information about positioning of piston 106 may include an indicated position that is different than a target position associated with a predetermined amount of medicine (e.g., a dose of medicine, without limitation). Such an indicated position may be ahead of, or behind, the target position. Ahead of the target position may indicate insufficient venting of working fluid at pneumatic drive system 116. Behind the target position may indicate venting of working fluid at pneumatic drive system 116 is occurring at the wrong time or indicate over venting of working fluid at pneumatic drive system 116.

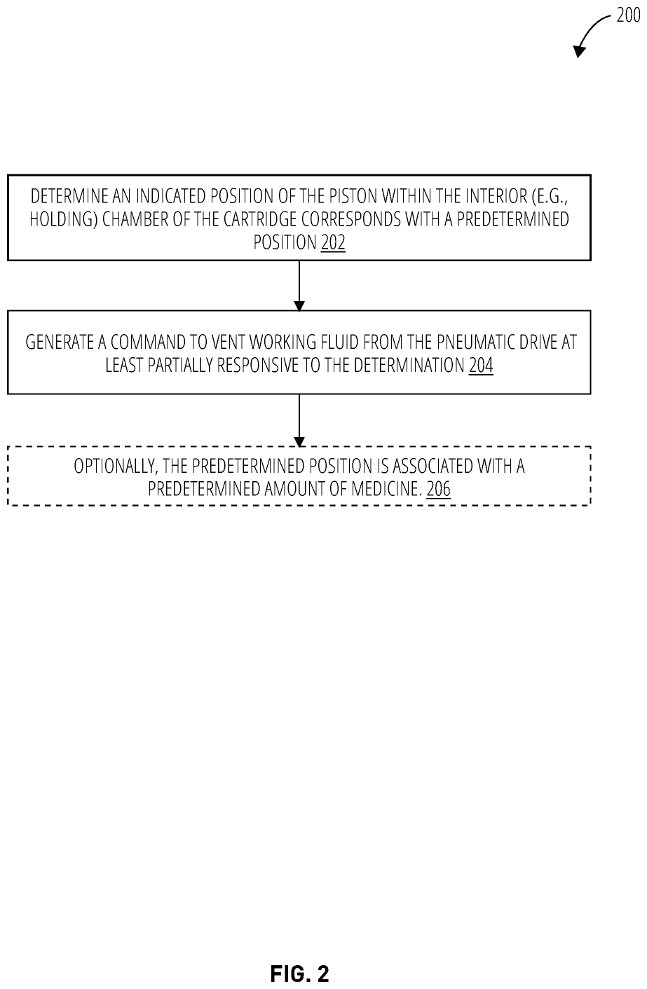

FIG. 2 illustrates an example process 200 controlling venting at pneumatic drive system 116 in accordance with one more examples. Process 200 may be performed, as a non-limiting example, by controller 134 of FIG. 1.

Although the example process 200 depicts a particular sequence of operations, the sequence may be altered without departing from the scope of the present disclosure. For example, some of the operations depicted may be performed in parallel or in a different sequence that does not materially affect the function of the process 200. In other examples, different components of an example device or system that implements the process 200 may perform functions at substantially the same time or in a specific sequence.

At operation 202, process 200 determines an indicated position of a piston (e.g., piston 106, without limitation) within an interior chamber of a cartridge (e.g., interior chamber 104 of cartridge 102, without limitation) corresponds with a predetermined position. The cartridge may be configured to hold medicine, such as insulin, in at least a portion of a space of the interior chamber of the cartridge.

At operation 204, process 200 generates a command to vent working fluid from the pneumatic drive at least partially responsive to the determination at operation 204. Venting working fluid reduces the urging force applied to the piston, and thus applied to any medicine, by the drive mechanism (e.g., drive mechanism 136, without limitation) at least to a degree insufficient to advance the piston within the interior chamber of the cartridge.

At optional operation 206 of process 200, the predetermined position is associated with a predetermined amount of medicine at operation 206. The predetermined position may be associated with delivery of a desired amount of medicine. More specifically, the predetermined position may be associated with displacement of a predetermined volume of medicine within a first space (e.g., first space 108, without limitation) of the interior chamber of the cartridge. For example, in a contemplated dispensing operation, the piston may arrive at the predetermined position after starting the dispensing operation at a different position (a starting position). Moving from the starting position to the predetermined position should displace a predetermined volume of medicine, the predetermined volume of medicine corresponding to a dose, a multiple of a dose, or a fraction of a dose. In a case where the medicine is insulin, the predetermined volume may correspond to a unit of insulin.

In one or more examples, the predetermined position utilized to generate a command to vent working fluid may correspond to an earlier position, arrived at just before a position associated with displacement of a desired amount of medicine. The earlier position may be chosen to allow for a partial displacement of a volume of medicine between the earlier position and the predetermined position that occurs while working fluid is vented from the pneumatic drive system.

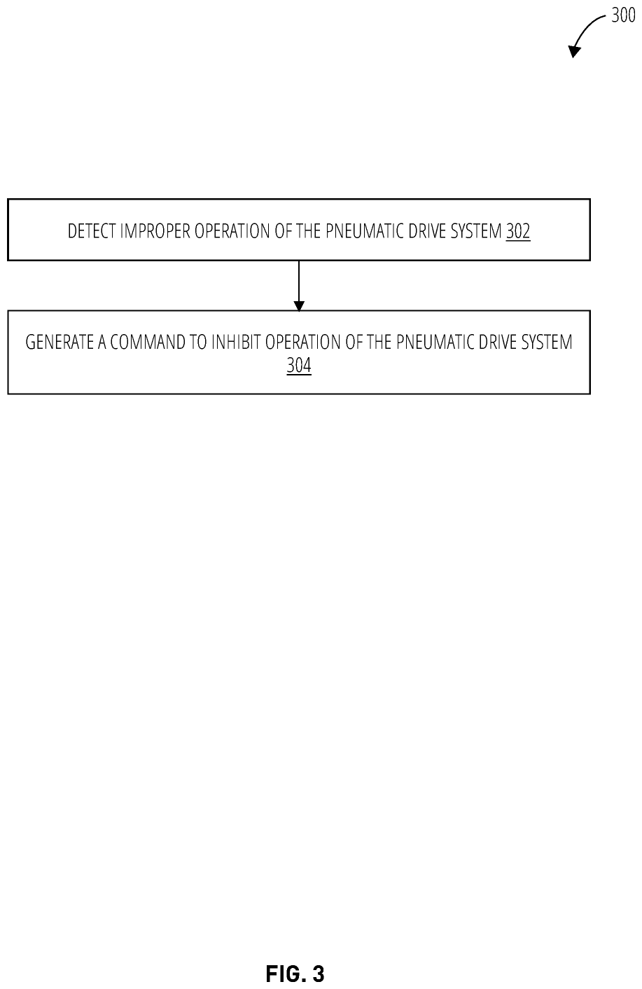

FIG. 3 illustrates an example process 300 for inhibiting operation of pneumatic drive system 116, in accordance with one or more examples. Process 300 may be performed, as a non-limiting example, by controller 134 of FIG. 1.

Although the example process 300 depicts a particular sequence of operations, the sequence may be altered without departing from the scope of the present disclosure. For example, some of the operations depicted may be performed in parallel or in a different sequence that does not materially affect the function of the process 300. In other examples, different components of an example device or system that implements the process 300 may perform functions at substantially the same time or in a specific sequence.

According to some examples, the method includes detecting improper operation of the pneumatic drive system at operation 302.

According to some examples, the method includes generating a command to inhibit operation of the pneumatic drive system at operation 304.

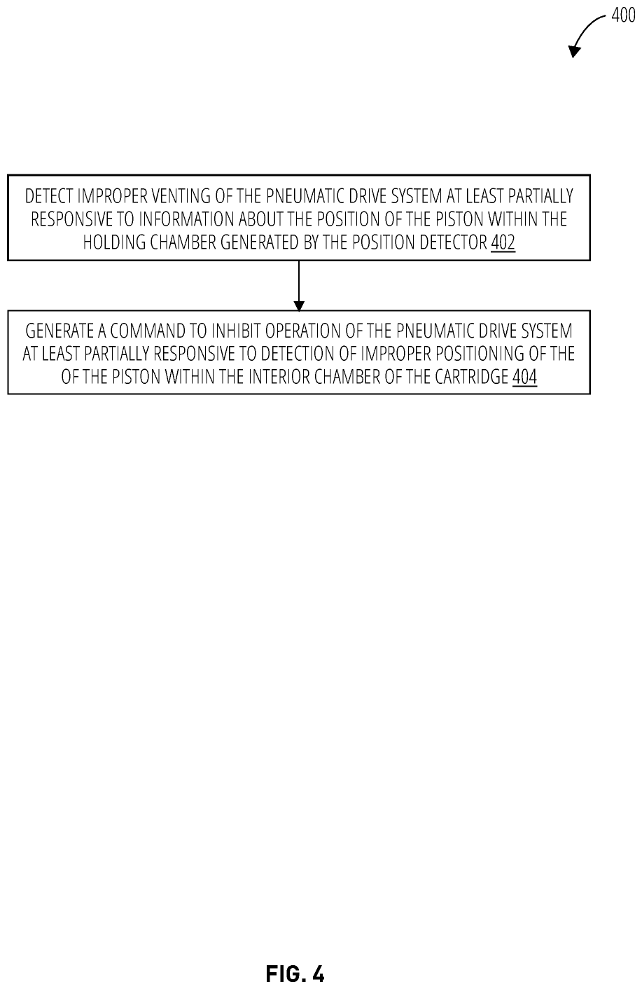

FIG. 4 illustrates an example process 400 for inhibiting operation of pneumatic drive system 116, in accordance with one or more examples. Process 400 may be performed, as a non-limiting example, by controller 134 of FIG. 1.

Although the example process 400 depicts a particular sequence of operations, the sequence may be altered without departing from the scope of the present disclosure. For example, some of the operations depicted may be performed in parallel or in a different sequence that does not materially affect the function of the process 400. In other examples, different components of an example device or system that implements the process 400 may perform functions at substantially the same time or in a specific sequence.

According to some examples, the method includes detecting improper venting of the pneumatic drive system at least partially responsive to information about the position of the piston within the holding chamber generated by the position detector at operation 402.

According to some examples, the method includes generating a command to inhibit operation of the pneumatic drive system at least partially responsive to detection of improper positioning of the of the piston within the interior chamber of the cartridge at operation 404.

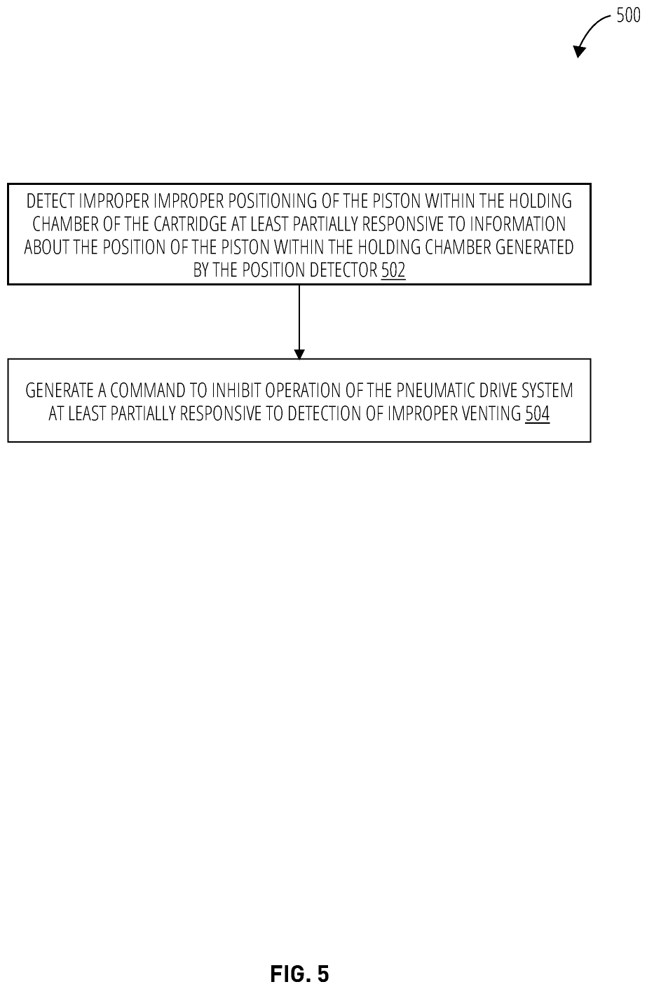

FIG. 5 illustrates an example process 500 for inhibiting operation of pneumatic drive system 116, in accordance with one or more examples. Process 500 may be performed, as a non-limiting example, by controller 134 of FIG. 1.

Although the example process 500 depicts a particular sequence of operations, the sequence may be altered without departing from the scope of the present disclosure. For example, some of the operations depicted may be performed in parallel or in a different sequence that does not materially affect the function of the process 500. In other examples, different components of an example device or system that implements the process 500 may perform functions at substantially the same time or in a specific sequence.

According to some examples, the method includes detecting improper positioning of the of the piston within the holding chamber of the cartridge at least partially responsive to information about the position of the piston within the holding chamber generated by the position detector at operation 502.

According to some examples, the method includes generating a command to inhibit operation of the pneumatic drive system at least partially responsive to detection of improper venting at operation 504.

One or more examples may be utilized in a variety of medication delivery devices. As a non-limiting example, system 100 may be utilized in a medication injection pen (also sometimes called an “injector”) such as an insulin injector pen.

Although some examples may appear settled or concrete, we apply pressure to encourage a fluid interpretation. Some examples may feel forced, however they “im-port” an understanding of “e-vents” discussed herein.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, without limitation) of the computing system. In some examples, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated.

As used in the present disclosure, the term “combination” with reference to a plurality of elements may include a combination of all the elements or any of various different subcombinations of some of the elements. For example, the phrase “A, B, C, D, or combinations thereof” may refer to any one of A, B, C, or D; the combination of each of A, B, C, and D; and any subcombination of A, B, C, or D such as A, B, and C; A, B, and D; A, C, and D; B, C, and D; A and B; A and C; A and D; B and C; B and D; or C and D.

Terms used in the present disclosure and especially in the appended claims (e.g., bodies of the appended claims, without limitation) are generally intended as “open” terms (e.g., the term “including” should be interpreted as “including, but not limited to,” the term “having” should be interpreted as “having at least,” the term “includes” should be interpreted as “includes, but is not limited to,” without limitation). As used herein, the term “each” means “some or a totality.” As used herein, the term “each and every” means a “totality.”

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more,” without limitation); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations, without limitation). Furthermore, in those instances where a convention analogous to “at least one of A, B, and C, without limitation” or “one or more of A, B, and C, without limitation” is used, in general such a construction is intended to include A alone, B alone, C alone, A and B together, A and C together, B and C together, or A, B, and C together, without limitation.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additional non-limiting examples include:

Example 1: A system, comprising: a cartridge including a piston configured to move within a holding chamber of the cartridge and urge medicine toward an interface between the holding chamber and a region external to the cartridge; a pneumatic drive system to actuate movement of the piston within the holding chamber; a position detector to generate information about a position of the piston within the holding chamber; and a controller to vent working fluid from the pneumatic drive system at least partially responsive to information about the position of the piston within the holding chamber generated by the position detector.

Example 2: The system according to Example 1, wherein the controller is configured to: determine an indicated position of the piston within the holding chamber of the cartridge corresponds with a predetermine position; and generate a command to vent working fluid from the pneumatic drive at least partially responsive to the determination.

Example 3: The system according to any of Examples 1 and 2, where the predetermined position is associated with a predetermined amount of medicine.

Example 4: The system according to any of Examples 1-3, wherein the pneumatic drive system comprises: a compressor to generate working fluid; a drive mechanism to apply force to the piston; a transfer system to provide working fluid to and from the drive mechanism; a port to permit escape of working fluid from the transfer system; and a valve to regulate flow of working fluid from the transfer system to the port.

Example 5: The system according to any of Examples 1-4, wherein the valve is configured to exhibit at least two states, the at least two states comprising an open state and a closed state, wherein the open state is associated with permitting flow of working fluid from the transfer system to the port, and wherein the closed state is associated with inhibiting or blocking flow of working fluid from the transfer system to the port.

Example 6: The system according to any of Examples 1-5, comprising a drive mechanism configured to, at least partially responsive to a command to vent working fluid, change a state of the valve from the open state to the closed state, and from the closed state to the open state.

Example 7: The system according to any of Examples 1-6, wherein the compressor includes: a tank; and a mechanical button coupled with a piston within the tank to add working fluid to the pneumatic drive system at least partially responsive to manual operation of the mechanical button.

Example 8: The system according to any of Examples 1-7, wherein the mechanical button is configured to move the piston within the tank to add working fluid to pneumatic drive system.

Example 9: The system according to any of Examples 1-8, wherein an added amount of working fluid responsive to a single operational cycle of the mechanical button is associated with a predetermined volume displacement of medicine within the holding chamber of the cartridge.

Example 10: The system according to any of Examples 1-9, wherein the predetermined volume displacement of medicine is associated with a predetermined amount of medicine.

Example 11: The system according to any of Examples 1-10, wherein a tank of the compressor is sized to limit an amount of working fluid in the tank to a predetermined maximum amount of working fluid.

Example 12: The system according to any of Examples 1-11, wherein the predetermined maximum amount of working fluid is associated with a predetermined maximum volume displacement of medicine within the holding chamber of the cartridge.

Example 13: The system according to any of Examples 1-12, wherein the drive mechanism includes a chamber of the cartridge behind the piston, the chamber to hold working fluid received via the transfer system.

Example 14: The system according to any of Examples 1-13, comprising an inhibitor to selectively inhibit operation of the pneumatic drive system.

Example 15: The system according to any of Examples 1-14, wherein the controller is configured to enable the inhibitor at least partially responsive to detection of improper venting of the pneumatic drive system.

Example 16: The system according to any of Examples 1-15, wherein the controller is configured to detect improper venting of the pneumatic drive system at least partially responsive to information about the position of the piston within the holding chamber generated by the position detector.

Example 17: The system according to any of Examples 1-16, wherein the controller is configured to enable the inhibitor at least partially responsive to detection of improper positioning of the of the piston within the holding chamber of the cartridge.

Example 18: The system according to any of Examples 1-17, wherein the controller to detect improper positioning of the of the piston within the holding chamber of the cartridge at least partially responsive to information about the position of the piston within the holding chamber generated by the position detector.

Example 19: The system according to any of Examples 1-18, wherein the position detector is configured to detect presence of the piston at one or more locations along the extent of the cartridge.

Example 20: The system according to any of Examples 1-19, wherein the position detector system includes: a signal detector; and a coupling system to movably couple the signal detector to an outside surface of the cartridge at one or more locations along the extent of the cartridge.

Example 21: The system according to any of Examples 1-20, wherein the position detector system includes an optical signal detector.

Example 22: The system according to any of Examples 1-21, wherein the position detector system includes a capacitive signal detector.

Example 23: The system according to any of Examples 1-22, wherein the position detector system includes an inductive signal detector.

Example 24: A method, comprising: determining an indicated position of a piston within an interior chamber of a cartridge corresponds with a predetermine position; and generating a command to vent working fluid from a pneumatic drive providing urging force to the piston at least partially responsive to the determination.

While the present disclosure has been described herein with respect to certain illustrated examples, those of ordinary skill in the art will recognize and appreciate that the present invention is not so limited. Rather, many additions, deletions, and modifications to the illustrated and described examples may be made without departing from the scope of the invention as hereinafter claimed along with their legal equivalents. In addition, features from one example may be combined with features of another example while still being encompassed within the scope of the invention as contemplated by the inventor.

What is claimed is:

1. A system, comprising:
a cartridge including a piston configured to move within a holding chamber of the cartridge and urge medicine toward an interface between the holding chamber and a region external to the cartridge;
a pneumatic drive system to actuate movement of the piston within the holding chamber;
a position detector to generate information about a position of the piston within the holding chamber; and
a controller to vent working fluid from the pneumatic drive system at least partially responsive to information about the position of the piston within the holding chamber generated by the position detector.

2. The system of claim 1, wherein the controller is configured to:
determine an indicated position of the piston within the holding chamber of the cartridge corresponds with a predetermine position; and
generate a command to vent working fluid from the pneumatic drive at least partially responsive to the determination.

3. The system of claim 2, where the predetermined position is associated with a predetermined amount of medicine.

4. The system of claim 1, wherein the pneumatic drive system comprises:
a compressor to generate working fluid;
a drive mechanism to apply force to the piston;
a transfer system to provide working fluid to and from the drive mechanism;
a port to permit escape of working fluid from the transfer system; and
a valve to regulate flow of working fluid from the transfer system to the port.

5. The system of claim 4, wherein the valve is configured to exhibit at least two states, the at least two states comprising an open state and a closed state, wherein the open state is associated with permitting flow of working fluid from the transfer system to the port, and wherein the closed state is associated with inhibiting or blocking flow of working fluid from the transfer system to the port.

6. The system of claim 5, comprising a drive mechanism configured to, at least partially responsive to a command to vent working fluid, change a state of the valve from the open state to the closed state, and from the closed state to the open state.

7. The system of claim 4, wherein the compressor includes:
a tank; and
a mechanical button coupled with a piston within the tank to add working fluid to the pneumatic drive system at least partially responsive to manual operation of the mechanical button.

8. The system of claim 7, wherein the mechanical button is configured to move the piston within the tank to add working fluid to pneumatic drive system.

9. The system of claim 8, wherein an added amount of working fluid responsive to a single operational cycle of the mechanical button is associated with a predetermined volume displacement of medicine within the holding chamber of the cartridge.

10. The system of claim 9, wherein the predetermined volume displacement of medicine is associated with a predetermined amount of medicine.

11. The system of claim 4, wherein a tank of the compressor is sized to limit an amount of working fluid in the tank to a predetermined maximum amount of working fluid.

12. The system of claim 11, wherein the predetermined maximum amount of working fluid is associated with a predetermined maximum volume displacement of medicine within the holding chamber of the cartridge.

13. The system of claim 4, wherein the drive mechanism includes a chamber of the cartridge behind the piston, the chamber to hold working fluid received via the transfer system.

14. The system of claim 1, comprising an inhibitor to selectively inhibit operation of the pneumatic drive system.

15. The system of claim 14, wherein the controller is configured to enable the inhibitor at least partially responsive to detection of improper venting of the pneumatic drive system.

16. The system of claim 15, wherein the controller is configured to detect improper venting of the pneumatic drive system at least partially responsive to information about the position of the piston within the holding chamber generated by the position detector.

17. The system of claim 14, wherein the controller is configured to enable the inhibitor at least partially responsive to detection of improper positioning of the of the piston within the holding chamber of the cartridge.

18. The system of claim 17, wherein the controller to detect improper positioning of the of the piston within the holding chamber of the cartridge at least partially responsive to information about the position of the piston within the holding chamber generated by the position detector.

19. The system of claim 1, wherein the position detector is configured to detect presence of the piston at one or more locations along the extent of the cartridge.

20. The system of claim 19, wherein the position detector system includes:
a signal detector; and
a coupling system to movably couple the signal detector to an outside surface of the cartridge at one or more locations along the extent of the cartridge.

21. The system of claim 1, wherein the position detector system includes an optical signal detector.

22. The system of claim 1, wherein the position detector system includes a capacitive signal detector.

23. The system of claim 1, wherein the position detector system includes an inductive signal detector.

24. A method, comprising:
determining an indicated position of a piston within an interior chamber of a cartridge corresponds with a predetermine position; and
generating a command to vent working fluid from a pneumatic drive providing urging force to the piston at least partially responsive to the determination.
